(19) United States
(12) Patent Application Publication
(10) Pub. No.: US 2007/0208257 A1
(43) Pub. Date: Sep. 6, 2007

(54) Lateral Viewing Optical Catheters

(76) Inventor: Simon M. Furnish, New York, NY (US)

Correspondence Address: PATTON BOGGS LLP, 8484 WESTPARK DRIVE, SUITE 900, MCLEAN, VA, 22102, US

(21) Appl. No.: 11/681,110

(22) Filed: Mar. 1, 2007

Related U.S. Application Data
(60) Provisional application No. 60/778,399, filed on Mar. 3, 2006.

(51) Int. Cl. A61B 5/02

(52) U.S. Cl. 600/479

(57) ABSTRACT

The present invention provides improved catheters in which lateral-viewing optical components extend radially outward from the catheter axis to optically interrogate blood vessel walls. One aspect of the invention provides a guidewire-compatible design for this type of catheter. In another aspect of invention, the lateral-viewing optical components are disposed on multiple branches that are collectively disposed within the lumen of an expandable balloon.

This application claims the benefit of U.S. provisional patent application Ser. No. 60/778,399 filed Mar. 3, 2006, which is incorporated by reference herein in its entirety.

### FIELD OF THE INVENTION

The invention relates generally to the field of vascular catheters having optical diagnostic capabilities.

### BACKGROUND OF INVENTION

Various optical modalities for diagnostically interrogating blood vessel walls to locate and characterize atherosclerotic lesions have been described. What is needed are improved catheter probes, particularly for the location and diagnosis of vulnerable plaque lesions. Vulnerable plaques, which are sometimes known as high-risk atherosclerotic plaques, include arterial atherosclerotic lesions characterized by a subluminal thrombotic lipid-rich pool of materials contained by a thin fibrous cap. Although vulnerable plaques are non-stenotic or nominally stenotic, it is believed that their rupture, resulting in the release of thrombotic contents, accounts for a significant fraction of adverse cardiac events.

U.S. Pat. No. 6,522,913 discloses systems and methods for visualizing tissue during diagnostic or therapeutic procedures that utilize a support structure that brings sensors into contact with the lumen wall of a blood vessel, and is incorporated by reference herein in its entirety.

U.S. Pat. No. 6,701,181 discloses multi-path optical catheters, and is incorporated by reference herein in its entirety.

U.S. Pat. No. 6,873,868 discloses multi-fiber catheter probe arrangements for tissue analysis or treatment, and is incorporated by reference herein in its entirety.

U.S. Pat. No. 6,949,072 discloses devices for vulnerable plaque detection, and is incorporated by reference herein in its entirety.

U.S. Publication No. 2002/0183622 discloses a fiber-optic apparatus and method for the optical imaging of tissue samples, and is incorporated by reference herein in its entirety.

U.S. Publication No. 2003/0125630 discloses catheter probe arrangements for tissue analysis by radiant energy delivery and radiant energy collection, and is incorporated by reference herein in its entirety.

U.S. Publication No. 2004/0204651 discloses infrared endoscopic balloon probes, and is incorporated by reference herein in its entirety.

U.S. Publication No. 2004/0260182 discloses intraluminal spectroscope devices with wall-contacting probes, and is incorporated by reference herein in its entirety.

U.S. Publication No. 2005/0054934 discloses an optical catheter with dual-stage beam redirector, and is incorporated by reference herein in its entirety.

U.S. Publication No. 2005/0075574 discloses devices for vulnerable plaque detection that utilize optical fiber temperature sensors, and is incorporated by reference herein in its entirety.

U.S. Publication No. 2005/0165315 discloses a side-firing fiber-optic array probe, and is incorporated by reference herein in its entirety.

### SUMMARY OF INVENTION

The present invention provides side/lateral-viewing catheters in which optical components for interrogating the walls of blood vessel lumens are disposed on rod elements that flex outward toward a blood vessel wall.

One embodiment of the invention provides a catheter apparatus for optically interrogating a blood vessel wall, that includes: (1) multiple optical probe rod elements (e.g., 2, 3, 4, 6, or 8) along a central shaft of the catheter that are radially extendable outward toward a blood vessel wall from an unextended configuration closer to the longitudinal (central) axis of the catheter; and (2) an expandable balloon collectively enclosing the multiple rod elements. The rod elements may each include an optical assembly for transmitting and receiving light from the vessel wall lateral to the axis of the catheter while the rod-elements contact or are near the wall. Each of the optical assemblies is in optical communication with at least one optical fiber that is in communication with a light source for illuminating the vessel wall and/or a detector for detecting light received from the vessel wall. The optical assemblies of each rod element may be disposed at or around the middle of a rod element or at or around whatever part of a rod element tends to extend most radially outward.

Another embodiment of the invention provides a catheter apparatus for optically interrogating a blood vessel wall that includes: (1) a rod element portion near the distal end of the catheter comprising multiple rod elements along a central shaft of the catheter and extendable radially outward toward a blood vessel wall from an unextended configuration closer to the longitudinal (central) axis of the catheter, wherein the rod elements each comprise an optical assembly for transmitting and receiving light from the vessel wall lateral to the axis of the catheter while the rod elements contact or are near the wall, where each of the optical assemblies is in optical communication with at least one optical fiber that is in communication with a light source for illuminating the vessel wall and/or a detector for detecting light received from the vessel wall; and (2) a tip portion of the catheter that extends from the distal end of the rod element portion to the distal end of the catheter, wherein a guidewire conduit or channel extends from within the central shaft of the rod element portion of the catheter distally through the tip portion of the catheter. The guidewire channel or conduit may, for example, open within the rod element portion of the catheter and at or near the distal end of the tip portion of the catheter.

The invention also provides methods for evaluating the condition of blood vessels using the optical catheter embodiments of the invention, which may, for example, include identifying, locating and/or characterizing atherosclerotic lesions in a blood vessel, such as an artery, using any of the optical catheter embodiments of the invention. A related embodiment of the invention provides methods for identifying, locating and/or characterizing vulnerable plaque lesions in a blood vessel, such as an artery, using any of the optical catheter embodiments of the invention.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

FIGS. 1A through 1D show various views of a lateral-viewing “basket-style” optical catheter according to the invention.

FIG. 2A is a cross-section of a blood vessel in which a four-probe optical catheter embodiment of the invention optically interrogates four non-overlapping fields of view. FIG. 2B shows a lipid profile for the four interrogated radial regions of the blood vessel shown in FIG. 2A.

FIGS. 3A through 3C show a basket-style optical catheter embodiment of the invention which is adapted to receive a guidewire.

FIGS. 4A through 4E show a balloon-enclosed embodiment of the invention in which the rod elements of the catheter are collectively enclosed within the lumen of a balloon that encloses the entire rod element portion of the catheter.

### DETAILED DESCRIPTION

The present invention provides side/lateral-viewing catheters in which optical components for interrogating the walls of blood vessel lumens are disposed on rods that can be flexed outward toward a blood vessel wall.

One embodiment of the invention provides catheter apparatus for optically interrogating a blood vessel wall, that includes: multiple optical probe rod elements (e.g., 2, 3, 4, 6, or 8) along a central shaft of the catheter and extendable radially outward toward a blood vessel wall from an unextended configuration closer to the longitudinal (central) axis of the catheter and an expandable balloon collectively enclosing the multiple rod elements. The rod elements may each include an optical assembly for transmitting and receiving light from the vessel wall lateral to the axis of the catheter while the rod-elements contact or are near the wall. Each of the optical assemblies is in optical communication with at least one optical fiber that is in communication with a light source for illuminating the vessel wall and/or a detector for detecting light received from the vessel wall. The optical assemblies of each rod element may be disposed at or around the middle of a rod element or at or around whatever part of a rod element tends to extend most radially outward.

The balloon may be shaped or configured to allow the passage of blood past the rod portion of the catheter while it is in its expanded, “working” state. The balloon may have a lobed structure so that each of the rod elements is disposed within a longitudinally oriented lobe. This helps to maintain a desired radial orientation of the rod elements in relation to each other. The rod elements may be at least approximately equally radially spaced about the axis of the catheter. A lobed balloon may be used to help maintain the rod elements in this orientation. The multiple rod elements may be located near the distal end of the catheter apparatus. A distal tip may be located distal to the rod element portion of the catheter. Relative motion of the distal ends and proximal ends of the rods may be used to radially flex the rods outward toward a lumen wall and to radially retract the rods toward the catheter axis. The lobed balloon may maintain its lobed shape in its expanded state so that blood can flow past the optical interrogation portion of the catheter during its operation.

In another embodiment, a multi-chambered balloon is used instead of a lobed balloon as described. The chambered balloon has a single proximal opening that is sealed against the catheter and a single distal opening that is sealed against the catheter. Between its ends, the balloon divides into a number of tubular balloon sections with each rod sitting within the lumen of one of the sections.

An advantage of the balloon embodiments of the invention is that the balloons in their expanded state clear blood out of the field of view of the optical assemblies. A compliant balloon that touches or very closely approximates the vessel wall may be used while the underlying optical assemblies of the rods themselves do not touch the vessel wall and are farther away from the vessel wall. With this configuration, the catheter can be rapidly moved through a blood vessel with reduced risk of injury (from contact with the rods) while the optical analysis is performed with reduced interference from blood in the field of view.

Another embodiment of the invention provides a catheter apparatus for optically interrogating a blood vessel wall that includes: (1) a rod element portion near the distal end of the catheter comprising multiple rod elements along a central shaft of the catheter and extendable radially outward toward a blood vessel wall from an unextended configuration closer to the longitudinal (central) axis of the catheter, wherein the rod elements each include an optical assembly for transmitting and receiving light from the vessel wall lateral to the axis of the catheter while the rod-elements contact or are near the wall and wherein each of the optical assemblies is in optical communication with at least one optical fiber that is in communication with a light source for illuminating the vessel wall and/or a detector for detecting light received from the vessel wall; and (2) a tip portion of the catheter that extends from the distal end of the rod element portion to the distal end of the catheter, wherein a guidewire conduit or channel extends from within the central shaft of the rod element portion of the catheter distally through the tip portion of the catheter. The guidewire channel or conduit may, for example, open within the rod element portion of the catheter and at or near the distal end of the tip portion of the catheter.

Any suitable sort of side/lateral-viewing optical assembly(ies) may be used and numerous sorts of side-viewing optics are known in the art. For example, a 45-deg (or other angle) mirror face or a prism can be used to laterally direct/redirect light from an optical fiber. Similarly, an optical fiber can be provided with an angularly faceted tip to direct and receive light that is off-axis with respect to the fiber. In one embodiment of the invention, the balloon catheter includes at least two rod elements having side viewing optical assemblies, each having a different radial field of view. Each side-viewing optical assembly may be associated with at least one optical fiber that longitudinally spans the body of the catheter from its proximal end to the side-viewing optical assembly so that light transmitted from the proximal end of the catheter, such as from a laser, can be directed to the blood vessel wall and light received from the blood vessel wall can be transmitted out of the catheter for analysis. The side-viewing optics may, for example, include four side-viewing optical assemblies, each radially separated from adjacent assemblies by 90-deg or approximately 90-deg and each having an at least substantially non-overlapping radial field of view with the other. Each of these side-viewing optical assemblies may be associated with at least one optical fiber that longitudinally spans the body of the catheter from its proximal end to the side viewing optical assembly so that light transmitted from the proximal end of the catheter, such as from a laser, can be directed to the blood vessel wall and light received from the blood vessel wall can be transmitted out of the catheter for analysis.

Various aspects of the present invention are further described below with reference to the attached drawings.

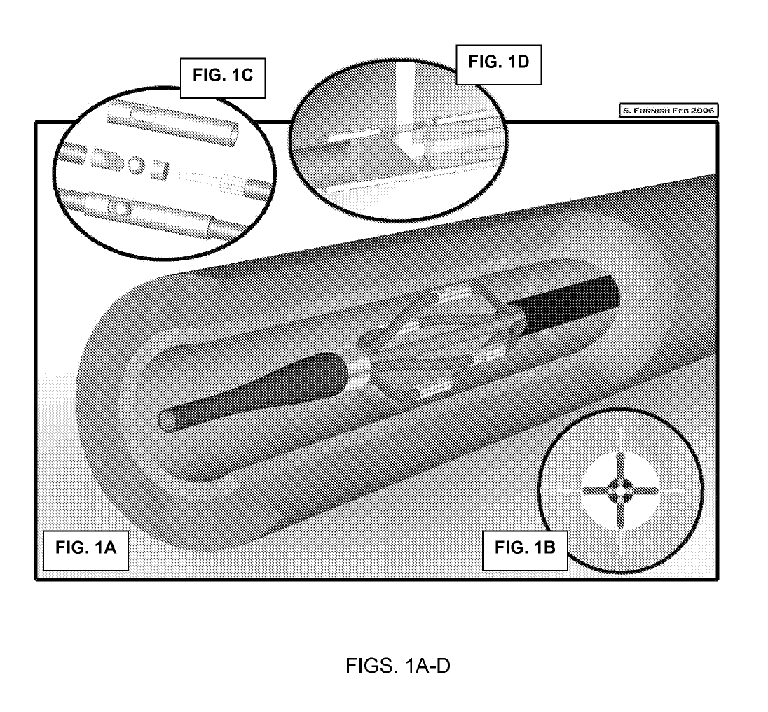

FIGS. 1A through 1D show various views of an embodiment of a lateral-viewing “basket-style” optical catheter according to the invention. FIG. 1A shows the basket-style optical catheter in an expanded working state in a blood vessel. The four rod elements containing lateral-viewing optical assemblies are flexibly expanded outward from the central shaft of the catheter and are contacting the vessel wall. In practice, the optical assembly parts of the rod element may be in actual contact with the wall or be near the wall. The rod elements may have a natural tendency to bow outward toward the wall. The radial extension of the rod elements may, for example, be controlled by relative movement of the tip section of the catheter. With the other elements remaining in position, drawing the tip section proximally (or similar relative movements) causes the rod elements to radially flex outward. The tip of the catheter is shown distal to the basket portion. FIG. 1B shows an end-on view of the device in the blood vessel which may, for example, be an artery, such as the coronary artery. The rod elements are shown in contact or close proximity with the vessel wall. The optical assemblies are disposed around the midpoint of each rod element. The white columns extending outward into the vessel wall represent the field of view of the optical assembly. As shown here, the fields of view of the four optical assemblies are not overlapping. FIG. 1C shows an exploded and assembled view of an embodiment of a rod-element. FIG. 1D is a cross sectional view of the side-viewing optical assembly component of the rod-element in communication with an optical fiber. As shown in FIGS. 1C and 1D, in this embodiment, the rod element consists of a proximal half and a distal half that are joined by a coupling sheath that surrounds the distal end of the proximal half of the rod element and the proximal end of the distal half of the rod element. As shown unassembled in FIG. 1C and assembled in FIG. 1D, arranged from left to right at the junction of the two halves of the rod element are a reflecting element, such as a 45-degree mirror face, a ball lens faceted to sit neatly against the reflecting element, a cylindrical optical element that may also be a filter, and the distal end of an optical fiber at the distal end of the proximal half of the rod element. As shown, the optical fiber may be centrally disposed within the tubular rod element. If the coupling sheath is insufficiently transparent, it may be provided with a viewing window, as shown in FIGS. 1C and 1D.

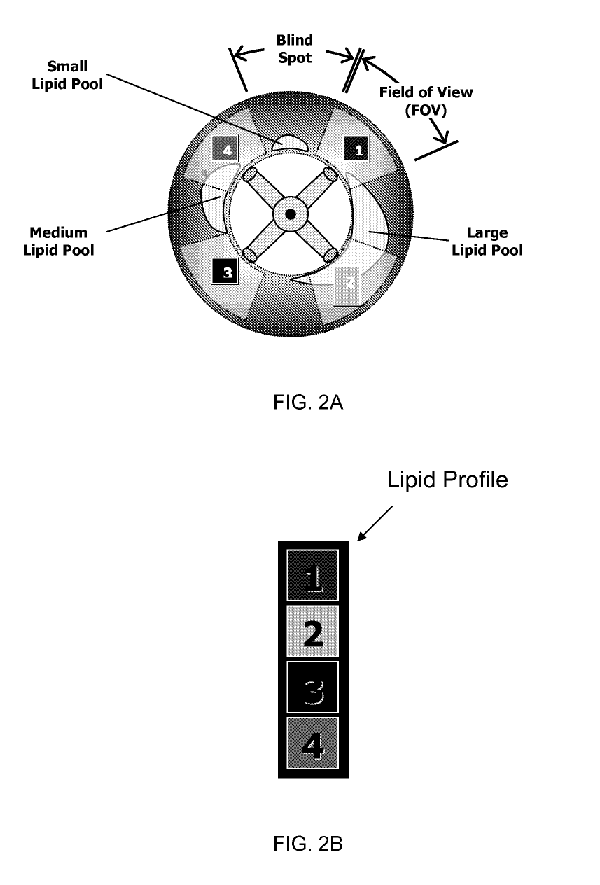

FIG. 2A is a cross-section of a blood vessel further illustrating how the fields of view of the rod-elements may be non-overlapping and how there may be blind spots about the inner circumference of a blood vessel that are not interrogated. Subluminal lipid pools of different sizes, as may be associated with vulnerable plaque, are shown. An optical reading or profile may be taken from each optical assembly at the location in the blood vessel. Based on the composition, such as the lipid composition, from each optical assembly, a radial profile indicative of the status of the blood vessel is obtained, as shown for lipids in FIG. 2B. Increased radial coverage can be obtained, for example, by using an increased number of rod elements and/or by using optical assemblies having larger fields of view.

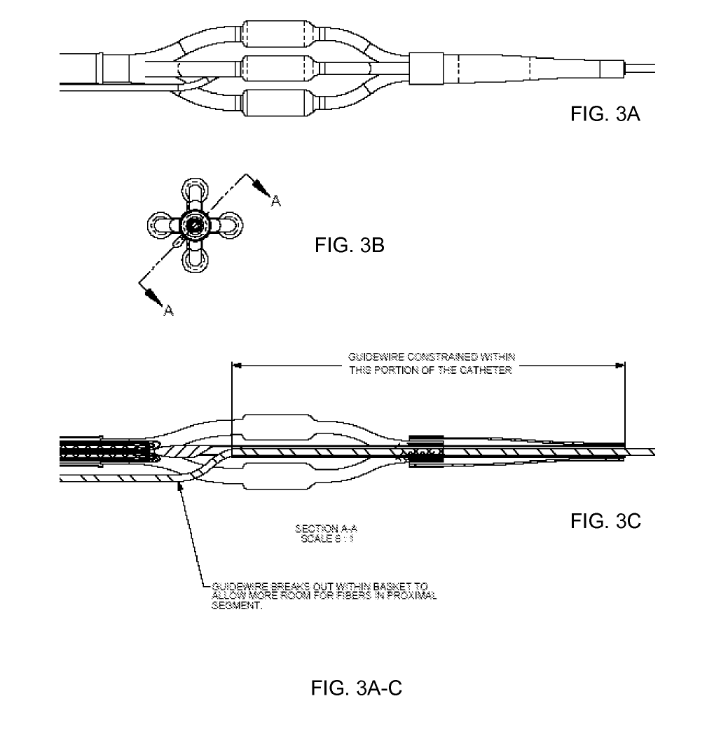

FIGS. 3A through 3C show a basket-style optical catheter embodiment of the invention which is adapted to receive a guidewire. This adaptation permits the catheter to be rapidly delivered to a site of interrogation such as the coronary artery over a pre-positioned guidewire. FIG. 3A is a solid profile depiction of the catheter assembly with the inserted catheter. FIG. 3B is an end-on view of the catheter assembly. As shown in FIG. 3C, an opening for the guidewire is present in the central shaft of the part of the catheter that has the rod elements. The opening may be positioned radially between the rod two elements to minimize interference with the rod elements. A guidewire conduit or channel extends from the opening in the shaft distally through the rod element portion into and through the tip portion of the catheter and opens again at the distal tip. This allows the catheter to be moved back and forth along a guidewire.

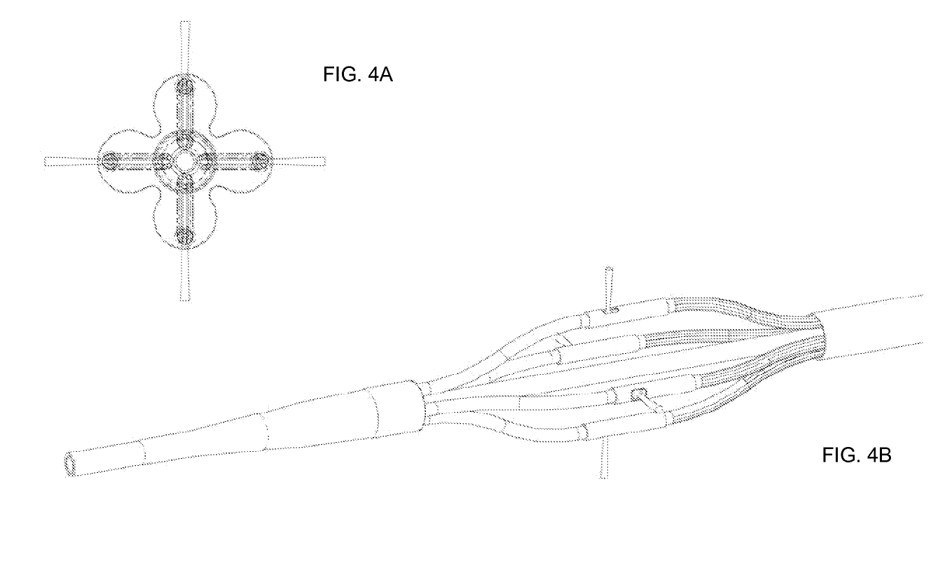

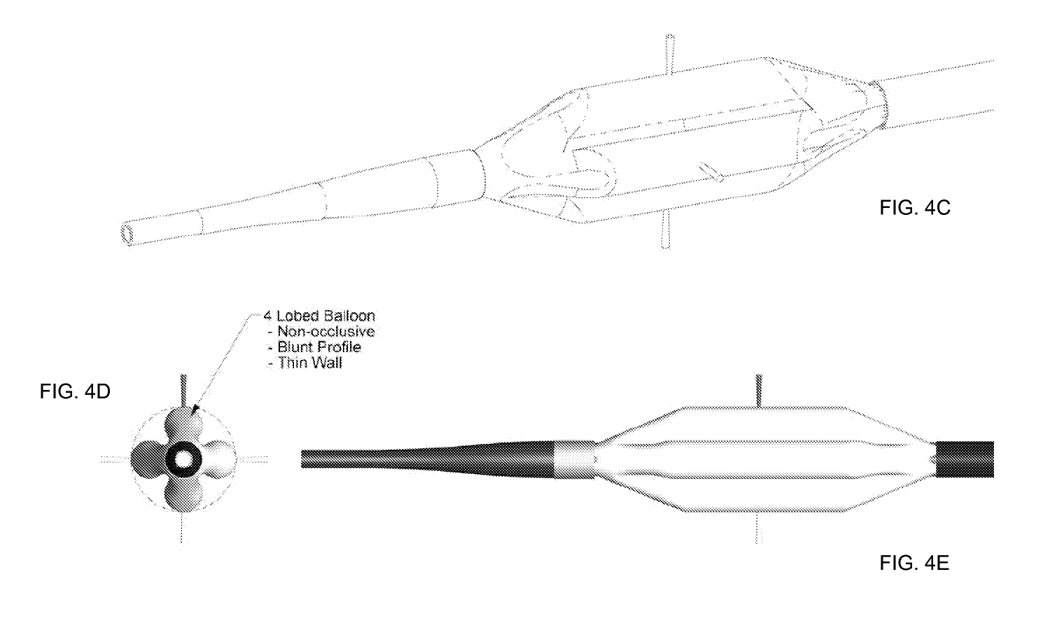

FIGS. 4A through 4E show a balloon-enclosed embodiment of the invention in which the rod elements of the catheter are collectively enclosed within the lumen of a balloon that encloses the entire rod element portion of the catheter. FIG. 4A is an end-on view of the distal portion of the balloon enclosed basket style catheter. The rods are shown in their radially expanded state enclosed by a balloon having four lobes, one for each rod. Each rod sits in a lobe of the balloon. This helps to keep the rods uniformly positioned about the radial axis. Space is also present adjacent to the lobes for blood to pass. FIG. 4B shows the basket-style optical catheter without its balloon cover. FIG. 4C shows a profile of the catheter enclosed in the four-lobe balloon. FIG. 4D shows a shaded (solid) end-on view and FIG. 4E shows a shaded (solid) profile view of the distal end of the catheter. The field of view of the optical assembly of each rod element is shown extending out from the catheter in each view.

Lobed balloons may, for example, be manufactured by molding techniques, such as those known in the art. For balloon-enclosed embodiments of the invention, the balloon may be at least partially made of a transparent balloon material. The balloon need only be at least substantially transparent to the particular wavelengths of light that are required to illuminate a target and receive back light for analysis, for a particular type of spectroscopy or optical analysis method. Thus, at least the part(s) of the balloon through which light is transmitted and received by the lateral-viewing optical assembly(ies) can be transparent to the extent required for a particular type of optical analysis. The balloon may also achieve a required level of transparency by having transparent windows through which light can pass that are not part of the general balloon material. The balloon may be reversibly and elastically deformed by the outward radial force of the rod elements and/or the balloon may be at least partially inflatable using a gas or liquid. The balloon may, for example, be a low-pressure inflatable balloon that is compliant. In one embodiment, the balloon is inflatable by 3 ATMs or less pressure. In a related embodiment, the balloon is inflatable by 2 ATMs or less pressure. In another embodiment, the balloon is inflatable by pressure between 1 ATM and 3 ATM.

Balloon materials may be selected according to the needs of a particular optical technique. Numerous polymers and polymer blends are available to select from. Suitable materials for the balloon coverings of the invention may, for example, include polyethylenes (such as PE, HDPE and LDPE), polyesters (such as PET), nylons and polyamides generally, fluoropolymers (such as PTFE and FEP), silicones and polyurethanes.

In an alternative balloon embodiment of the invention, the rods and/or optical assemblies are not outwardly expandable. Instead they remain at or near the central axis of the catheter. The optical assemblies may be disposed on or within rods or, for example, be collectively disposed on a single central shaft. A balloon enclosing the optical assemblies is expanded to or near a blood vessel wall to clear away blood for the optical interrogation of the tissue. A transparent gas or liquid may be used to expand the balloon. A conduit for the fluid may be provided down the length of the catheter and open into the lumen of the balloon in order to allow for inflation and deflation of the balloon In one variation, rather than using multiple optical assemblies to radially interrogate a lumen wall, at least one rotating lateral-viewing optical assembly is used to radially scan the lumen wall.

The invention is not limited by the optical method used to interrogate and diagnosis the condition of a blood vessel wall. Multiple methods may also be used. Suitable methods include, but are not limited to, low-resolution and high-resolution Raman spectroscopy, fluorescence spectroscopy, such as time-resolved laser-induced fluorescence spectroscopy, and laser speckle spectroscopy.

The invention also provides methods for evaluating the condition of blood vessels using the optical catheter embodiments of the invention, which may, for example, include identifying, locating and/or characterizing atherosclerotic lesions in a blood vessel, such as an artery, using any of the optical catheter embodiments of the invention. A related embodiment of the invention provides methods for identifying, locating and/or characterizing vulnerable plaque lesions in a blood vessel, such as an artery, using any of the optical catheter embodiments of the invention. One embodiment of the invention is a method for diagnosing and/or locating one or more vulnerable plaque lesions in a blood vessel, such as a coronary artery of a subject, using a catheter as described herein to optically evaluate the properties of a vessel wall at one more locations along the vessel. Any of the methods of evaluating the condition of a blood vessel using an optical catheter according to the invention may include moving the catheter laterally within a blood vessel to optically interrogate the blood vessel wall at different lateral positions. Optical sampling of the vessel wall be performed while the optical catheter is moving laterally within a blood vessel and/or while it is stopped at a lateral position within the vessel.

Differentially diagnosing, identifying and/or determining the location of a vulnerable plaque in a blood vessel of a patient can be performed by any method or combination of methods. For example, catheter-based systems and methods for diagnosing and locating vulnerable plaques can be used, such as those employing optical coherent tomography (“OCT”) imaging, temperature sensing for temperature differences characteristic of vulnerable plaque versus healthy vasculature, labeling/marking vulnerable plaques with a marker substance that preferentially labels such plaques, infrared elastic scattering spectroscopy, and infrared Raman spectroscopy (IR inelastic scattering spectroscopy). U.S. Publication No. 2004/0267110 discloses a suitable OCT system and is hereby incorporated by reference herein in its entirety. Raman spectroscopy-based methods and systems are disclosed, for example, in: U.S. Pat. Nos. 5,293,872; 6,208,887; and 6,690,966; and in U.S. Publication No. 2004/0073120, each of which is hereby incorporated by reference herein in its entirety. Infrared elastic scattering based methods and systems for detecting vulnerable plaques are disclosed, for example, in U.S. Pat. No. 6,816,743 and U.S. Publication No. 2004/0111016, each of which is hereby incorporated by reference herein in its entirety. Time-resolved laser-induced fluorescence methods for characterizing atherosclerotic lesions are disclosed in U.S. Pat. No. 6,272,376, which is incorporated by reference herein in its entirety.

Temperature sensing based methods and systems for detecting vulnerable plaques are disclosed, for example, in: U.S. Pat. Nos. 6,450,971; 6,514,214; 6,575,623; 6,673,066; and 6,694,181; and in U.S. Publication No. 2002/0071474, each of which is hereby incorporated by reference herein in its entirety. A method and system for detecting and localizing vulnerable plaques based on the detection of biomarkers is disclosed in U.S. Pat. No. 6,860,851, which is hereby incorporated by reference herein in its entirety.

The invention also provides an integrated system for evaluating the status of a blood vessel wall, for example, for diagnosing and/or locating vulnerable plaque lesions, that includes an optical balloon catheter according to the invention, in communication with a light source such as a laser for illuminating a target region of a blood vessel via the catheter and a light analyzer, such as a spectroscope, for analyzing the properties of light received from the target region via the catheter. One or more computers, or computer processors generally working in conjunction with computer accessible memory under the control of computer instructions, e.g., software, may be part of the system for controlling the system and/or for analyzing information obtained by the system.

Each of the patents and other publications cited in this disclosure is incorporated by reference in its entirety.

Although the foregoing description is directed to the preferred embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above.

What is claimed is:

1. A catheter apparatus for optically interrogating a blood vessel wall, comprising:
multiple rod elements disposed along a central shaft of the catheter and extendable radially outward toward a blood vessel wall from an unextended configuration closer to the central axis of the catheter, wherein the rod elements each comprise an optical assembly for transmitting and receiving light from the vessel wall lateral to the central axis of the catheter while the rod-elements contact or are near the wall and wherein each of the optical assemblies is in optical communication with at least one optical fiber that is in communication with a light source for illuminating the vessel wall and/or a detector for detecting light received from the vessel wall; and
an expandable balloon collectively enclosing the multiple rod elements.

2. The apparatus of claim 1, wherein the balloon is configured to permit the passage of blood in its expanded state.

3. The apparatus of claim 1, wherein the balloon has a lobed structure and each of the rod elements is disposed within a separate lobe of the lobed structure.

4. The apparatus of claim 3, wherein the balloon is configured to permit the passage of blood in its expanded state.

5. The apparatus of claim 1, wherein the rod elements are at least approximately equally radially spaced about the axis of the catheter.

6. The apparatus of claim 3, wherein the rod elements are at least approximately equally radially spaced about the central axis of the catheter.

7. The apparatus of claim 1, wherein the multiple rod elements are located near the distal end of the catheter apparatus.

8. A catheter apparatus for optically interrogating a blood vessel wall, comprising:
a rod element portion near the distal end of the catheter comprising multiple rod elements along a central shaft of the catheter and extendable radially outward toward a blood vessel wall from an unextended configuration closer to the central axis of the catheter, wherein the rod elements each comprise an optical assembly for transmitting and receiving light from the vessel wall lateral to the central axis of the catheter while the rod elements contact or are near the wall and wherein each of the optical assemblies is in optical communication with at least one optical fiber that is in communication with a light source for illuminating the vessel wall and/or a detector for detecting light received from the vessel wall; and
a tip portion of the catheter that extends from the distal end of the rod element portion to the distal end of the catheter,
wherein a guidewire conduit or channel extends from within the central shaft of the rod element portion of the catheter distally through the tip portion of the catheter.

9. The apparatus of claim 8, wherein the guidewire channel or conduit opens within the rod element portion of the catheter and at or near the distal end of the tip portion of the catheter.

10. The apparatus of claim 9, wherein the guidewire channel or conduit extends though the center of the tip portion of the catheter and exits the center of the distal end of the tip portion.
